(19) United States
(12) Patent Application Publication
(10) Pub. No.: US 2026/0115452 A1
(43) Pub. Date: Apr. 30, 2026

(54) METHODS AND SYSTEMS FOR DETECTING A SUCTION EVENT ASSOCIATED WITH A HEART PUMP

(71) Applicant: Abiomed, Inc., Danvers, MA (US)

(72) Inventors: Mohammed Alwatban, Danvers, MA (US); Sanket Yograj Thakare, Danvers, MA (US); Yuka Akiyama, Danvers, MA (US); Yuting Zhang, Danvers, MA (US)

(73) Assignee: Abiomed, Inc., Danvers, MA (US)

(21) Appl. No.: 19/357,571

(22) Filed: Oct. 14, 2025

Related U.S. Application Data
(60) Provisional application No. 63/707,378, filed on Oct. 15, 2024.

(51) Int. Cl. A61M 60/585; A61M 60/13; A61M 60/216; A61M 60/546; A61M 60/554

(52) U.S. Cl. CPC A61M 60/585; A61M 60/13; A61M 60/216; A61M 60/546; A61M 60/554; A61M 2205/18; A61M 2205/581; A61M 2205/583

(57) ABSTRACT

Methods and apparatus for detecting a suction event associated with operation of a heart pump are provided. In one example, the method includes receiving a motor current signal associated with a motor of the heart pump, receiving a pressure signal associated with the heart pump, determining a pump flow signal based on the pressure signal, detecting, using a computer processor, a suction event, and displaying, when a suction event is detected, an indication of the suction event on a user interface associated with the heart pump. Detecting a suction event may be based, at least in part on a first value of the motor current signal within a first time window being a first threshold amount less than a baseline motor current signal value, and a first value of the pump flow signal within the first time window being a second threshold amount less than a baseline pump flow value.

### CROSS-REFERENCE TO RELATED APPLICATIONS

This application claims the benefit under 35 U.S.C. § 119 (e) to U.S. Provisional Patent Application No. 63/707,378, filed Oct. 15, 2024, and titled, “METHODS AND SYSTEMS FOR DETECTING A SUCTION EVENT ASSOCIATED WITH A HEART PUMP,” the entire contents of which is incorporated herein by reference in its entirety.

### FIELD OF THE INVENTION

This disclosure relates to techniques for detecting a suction event during operation of a heart pump.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span one or more heart valves. The intravascular blood pump is typically disposed at the end of the catheter. Once in position, the pump may be used to assist the heart and pump blood through the circulatory system and, therefore, temporarily reduce load on the patient's heart, such as to enable the heart to recover after a heart attack. An exemplary intravascular blood pump is available from ABIOMED, Inc., Danvers, MA under the tradename Impella® heart pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. In some instances, the controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

An intravascular blood pump designed for right heart assistance can extend through the pulmonary valve and into the pulmonary artery to expel blood into the pulmonary artery. In some instances, a partial or complete obstruction of the flow through the blood pump may cause a compromised pump function, resulting in reduced patient support. The obstructed flow may be due to ingestion of biomaterial into the pump leading to a permanent obstruction of the flow or a suction event leading to a reversable obstruction of the pump inlet. In the case of a suction event, a healthcare provider or other user may attempt to reposition the blood pump or take some other corrective action to improve the flow through the pump. Described herein are systems and methods for detecting a suction event for a blood pump by analyzing signals associated with operation of the pump. Although at least some of the techniques described herein are used to detect suction events in a blood pump configured to provide right heart support, it should be appreciated that one or more of the techniques may also be used to detect suction events in a blood pump inserted across the aortic valve in the left side of the heart.

In some embodiments, a method of detecting a suction event associated with operation of a heart pump is provided. The method includes receiving a motor current signal associated with a motor of the heart pump, receiving a pressure signal associated with the heart pump, determining a pump flow signal based on the pressure signal, detecting, using a computer processor, a suction event. Detecting the suction event is based, at least in part on a first value of the motor current signal within a first time window being a first threshold amount less than a baseline motor current signal value, and a first value of the pump flow signal within the first time window being a second threshold amount less than a baseline pump flow value. The method further includes displaying, when a suction event is detected, an indication of the suction event on a user interface associated with the heart pump.

In one aspect, the pressure signal comprises a differential pressure signal across at least one valve of a heart of a patient. In another aspect, determining the pump flow signal based on the pressure signal comprises accessing stored data associating pump flow values to differential pressure signal values, and determining the pump flow signal based on stored data. In another aspect, the first value of the motor current signal within the first time window is a mean value of the motor current signal within the first time window. In another aspect, the first value of the pump flow signal within the first time window is a mean flow value within the first time window. In another aspect, the heart pump is positioned across a valve in a right side of a heart of a patient.

In another aspect, the method further includes determining, based on the motor current signal, the baseline motor current signal value, wherein the baseline motor current signal value is determined within at least one second time window before the first time window, and determining, based on the pump flow signal, the baseline pump flow value, wherein the baseline pump flow value is determined within the at least one second time window. In another aspect, the first threshold amount is a first threshold percentage of the baseline motor current signal value. In another aspect, the second threshold amount is a second threshold percentage of the baseline pump flow value. In another aspect, the method further includes setting the first threshold amount and the second threshold amount based, at least in part, on a speed of the heart pump.

In another aspect, the method further includes detecting, using the computer processor, whether one or more exit criteria for the suction event are satisfied, and displaying, when the one or more exit criteria for the suction event are satisfied, an indication that the suction event has ended on the user interface associated with the heart pump. In another aspect, detecting whether one or more exit criteria for the suction event are satisfied comprises detecting that the one or more exit criteria are satisfied when a second value of the motor current signal within a second time window is more than a third threshold amount greater than the first value of motor current signal, and a second value of the pump flow signal within the second time window is more than a fourth threshold amount greater than the first value of the pump flow signal. In another aspect, the third threshold amount is a percentage of the first value of the motor current signal. In another aspect, the fourth threshold amount is a percentage of the first value of the pump flow signal. In another aspect, the first threshold amount and the third threshold amount are different. In another aspect, the second threshold amount and the fourth threshold amount are different. In another aspect, displaying, when a suction event is detected, an indication of the suction event on a user interface associated with the heart pump comprises displaying an alarm on the user interface, and displaying, when the one or more exit criteria for the suction event are satisfied, an indication that the suction event has ended on the user interface associated with the heart pump comprises removing the alarm from the user interface. In another aspect, the alarm comprises a visual alarm and/or an audio alarm.

In some embodiments, a controller for a heart pump system is provided. The controller includes at least one hardware processor configured to receive a motor current signal associated with a motor of a heart pump of the heart pump system, receive a pressure signal associated with the heart pump, determine a pump flow signal based on the pressure signal, detect a suction event, and display, when a suction event is detected, an indication of the suction event on a user interface associated with the heart pump system. Detecting a suction event may be based, at least in part, on a first value of the motor current signal within a first time window being a first threshold amount less than a baseline motor current signal value, and a first value of the pump flow signal within the first time window being a second threshold amount less than a baseline pump flow value.

In one aspect, the pressure signal comprises a differential pressure signal across at least one valve of a heart of a patient. In another aspect, determining the pump flow signal based on the pressure signal comprises accessing stored data associating pump flow values to differential pressure signal values, and determining the pump flow signal based on stored data. In another aspect, the first value of the motor current signal within the first time window is a mean value of the motor current signal within the first time window. In another aspect, the first value of the pump flow signal within the first time window is a mean flow value within the first time window. In another aspect, the heart pump is positioned across a valve in a right side of a heart of a patient.

In another aspect, the at least one hardware processor is further configured to determine, based on the motor current signal, the baseline motor current signal value, wherein the baseline motor current signal value is determined within at least one second time window before the first time window, and determining, based on the pump flow signal, the baseline pump flow value, wherein the baseline pump flow value is determined within the at least one second time window. In another aspect, the first threshold amount is a first threshold percentage of the baseline motor current signal value. In another aspect, the second threshold amount is a second threshold percentage of the baseline pump flow value. In another aspect, the at least one hardware processor is further configured to set the first threshold amount and the second threshold amount based, at least in part, on a speed of the heart pump.

In another aspect, the at least one hardware processor is further configured to detect whether one or more exit criteria for the suction event are satisfied, and display, when the one or more exit criteria for the suction event are satisfied, an indication that the suction event has ended on the user interface associated with the heart pump. In another aspect, detecting whether one or more exit criteria for the suction event are satisfied comprises detecting that the one or more exit criteria are satisfied when a second value of the motor current signal within a second time window is more than a third threshold amount greater than the first value of motor current signal, and a second value of the pump flow signal within the second time window is more than a fourth threshold amount greater than the first value of the pump flow signal. In another aspect, the third threshold amount is a percentage of the first value of the motor current signal. In another aspect, the fourth threshold amount is a percentage of the first value of the pump flow signal. In another aspect, the first threshold amount and the third threshold amount are different. In another aspect, the second threshold amount and the fourth threshold amount are different. In another aspect, displaying, when a suction event is detected, an indication of the suction event on a user interface associated with the heart pump comprises displaying an alarm on the user interface, and displaying, when the one or more exit criteria for the suction event are satisfied, an indication that the suction event has ended on the user interface associated with the heart pump comprises removing the alarm from the user interface. In another aspect, the alarm comprises a visual alarm and/or an audio alarm.

In some embodiments, a heart pump system is provided. The heart pump system includes a heart pump including a motor and a pressure sensor configured to sense a pressure within a portion of a heart of a patient, and a controller. The controller is configured to determine a pump flow signal based on a pressure signal sensed by the pressure sensor, detect a suction event, and display, when a suction event is detected, an indication of the suction event on a user interface associated with the heart pump system. The controller is configured to detect the suction event based, at least in part on a first value of a motor current signal associated with the motor within a first time window being a first threshold amount less than a baseline motor current signal value, and a first value of the pump flow signal within the first time window being a second threshold amount less than a baseline pump flow value.

In some embodiments, a method of detecting a suction event associated with operation of a heart pump is provided. The method includes receiving a motor current signal associated with a motor of the heart pump, receiving a pressure signal associated with the heart pump, determining within a first time window of the motor current signal, a pulsatility value, determining within the first time window of the pressure signal a pressure value, detecting, using a computer processor, a suction event when the pulsatility value is greater than a first threshold value and the pressure value is less than a second threshold value, and displaying, when a suction event is detected, an indication of the suction event on a user interface associated with the heart pump.

In one aspect, the method further includes filtering the motor current signal to generate a filtered motor current signal, wherein determining the pulsatility value comprises determining the pulsatility value using the filtered motor current signal. In another aspect, filtering the motor current signal comprises filtering the motor current signal with an infinite impulse response (IIR) filter. In another aspect, determining the pulsatility value comprises determining a pulsatility index according to: max(MC_win)−min(MC_win)/mean(MC_win), wherein MC_win represents values of the motor current signal within the first time window.

In another aspect, determining the pressure value comprises determining a minimum value of the pressure signal within the first time window. In another aspect, the pressure signal is an optical pressure signal. In another aspect, the heart pump is positioned across a valve in a right side of a heart of a patient. In another aspect, the method further includes setting the first threshold value and the second threshold value based, at least in part, on a speed of the heart pump. In another aspect, the method further includes detecting, using the computer processor, whether one or more exit criteria for the suction event are satisfied, and displaying, when the one or more exit criteria for the suction event are satisfied, an indication that the suction event has ended on the user interface associated with the heart pump. In another aspect, displaying, when a suction event is detected, an indication of the suction event on a user interface associated with the heart pump comprises displaying an alarm on the user interface, and displaying, when the one or more exit criteria for the suction event are satisfied, an indication that the suction event has ended on the user interface associated with the heart pump comprises removing the alarm from the user interface. In another aspect, the alarm comprises a visual alarm and/or an audio alarm.

In some embodiments, a controller for a heart pump system is provided. The controller includes at least one hardware processor configured to receive a motor current signal associated with a motor of the heart pump, receive a pressure signal associated with the heart pump, determine within a first time window of the motor current signal, a pulsatility value, determine within the first time window of the pressure signal a pressure value, detect a suction event when the pulsatility value is greater than a first threshold value and the pressure value is less than a second threshold value, and display, when a suction event is detected, an indication of the suction event on a user interface associated with the heart pump.

In one aspect, the at least one hardware processor is further configured to filter the motor current signal to generate a filtered motor current signal, wherein determining the pulsatility value comprises determining the pulsatility value using the filtered motor current signal. In another aspect, filtering the motor current signal comprises filtering the motor current signal with an infinite impulse response (IIR) filter. In another aspect, determining the pulsatility value comprises determining a pulsatility index according to: max(MC_win)−min(MC_win)/mean(MC_win), wherein MC_win represents values of the motor current signal within the first time window.

In another aspect, determining the pressure value comprises determining a minimum value of the pressure signal within the first time window. In another aspect, the pressure signal is an optical pressure signal. In another aspect, the heart pump is positioned across a valve in a right side of a heart of a patient. In another aspect, the at least one hardware processor is further configured to set the first threshold value and the second threshold value based, at least in part, on a speed of the heart pump. In another aspect, the at least one hardware processor is further configured to detect, using the computer processor, whether one or more exit criteria for the suction event are satisfied, and display, when the one or more exit criteria for the suction event are satisfied, an indication that the suction event has ended on the user interface associated with the heart pump. In another aspect, displaying, when a suction event is detected, an indication of the suction event on a user interface associated with the heart pump comprises displaying an alarm on the user interface, and displaying, when the one or more exit criteria for the suction event are satisfied, an indication that the suction event has ended on the user interface associated with the heart pump comprises removing the alarm from the user interface. In another aspect, the alarm comprises a visual alarm and/or an audio alarm.

In some embodiments, a heart pump system is provided. The heart pump system includes a heart pump including a motor and a pressure sensor configured to sense a pressure within a portion of a heart of a patient, and a controller. The controller is configured to determine within a first time window of a motor current signal associated with the motor, a pulsatility value, determine within the first time window of pressure signal sensed by the pressure sensor, a pressure value, detect a suction event when the pulsatility value is greater than a first threshold value and the pressure value is less than a second threshold value, and display, when a suction event is detected, an indication of the suction event on a user interface associated with the heart pump.

### BRIEF DESCRIPTION OF DRAWINGS

FIG. 1A shows an illustrative cardiac support device that may be used with some embodiments.

FIG. 1B shows an illustrative cardiac support system that includes the cardiac support device of FIG. 1A.

FIG. 2 shows plots of example signals associated with operation of a cardiac support device, in accordance with some embodiments.

FIG. 3 is a flowchart of a process for detecting a suction event, in accordance with some embodiments.

FIG. 4 shows plots of example signals associated with operation of a cardiac support device, in accordance with some embodiments.

FIG. 5 is a flowchart of a process for detecting a suction event, in accordance with some embodiments.

### DETAILED DESCRIPTION

A cardiac support device (e.g., an intravascular blood pump) may include an impeller that may be controlled to pump blood from an inlet on a first side of a valve to an outlet on a second side of the valve, thereby at least partially replacing normal heart function in the patient. In some instances, the flow of blood through the cardiac support device may be reduced when the inlet of the pump is obstructed. For instance, when the inlet is placed close to the wall of a vessel, the tissue along the vessel wall may be pulled by the suction of the pump into the inlet, thereby creating a pump blockage and reducing blood flow through the device. Due to the reduced support being provided to a patient when such suction events occur, a healthcare provider or other user of a heart pump may be provided with an indication of the suction event so that they may perform a corrective action (e.g., repositioning the heart pump) to improve blood flow thorough the device. The inventors have recognized and appreciated that an analysis of signals associated with the operation of the cardiac support device may be used to automatically and reliably detect suction events, thereby providing users with useful information to enable them to take appropriate corrective actions.

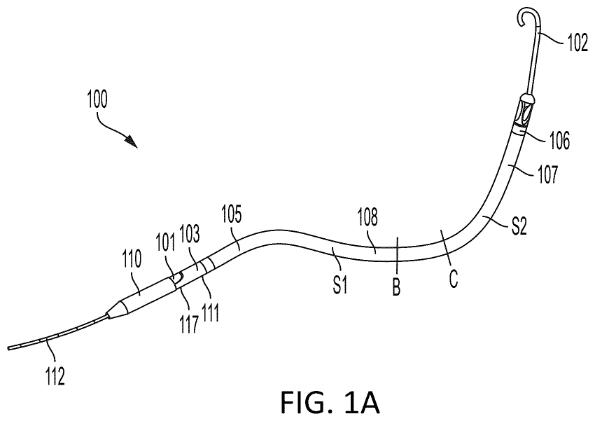

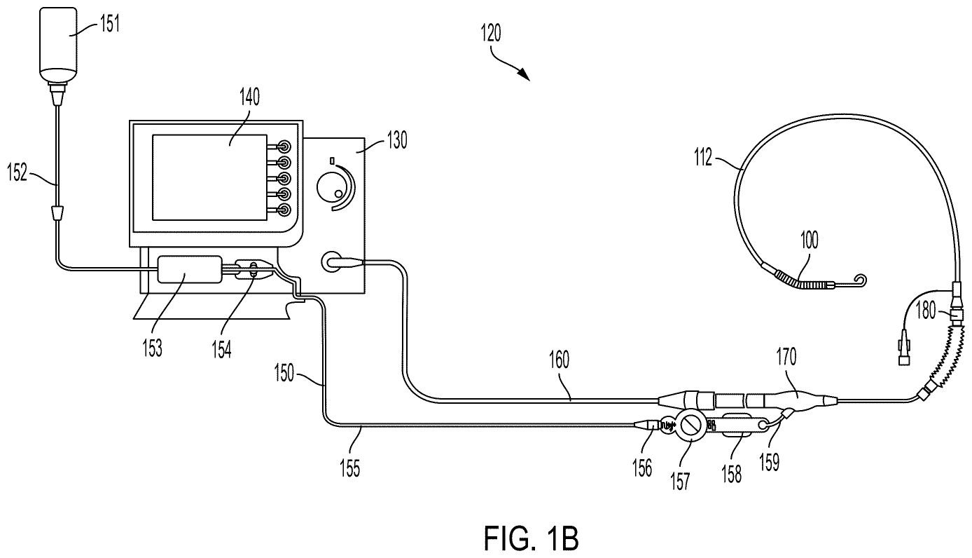

FIG. 1A shows an illustrative embodiment of a blood pump assembly 100 according to the present disclosure. The blood pump assembly 100 may include a pump 101, a pump housing 103, a proximal end 105, a distal end 107, a cannula 108, an impeller (not shown), an atraumatic extension 102, a catheter 112, an inlet area 110, an outlet area 106, and blood exhaust apertures 117. The catheter 112 may be connected to the inlet area 110 of the cannula 108 in some embodiments. The inlet area 110 may be located near the proximal end 105 of the cannula, and the outlet area 106 may be located toward the distal end 107 of the cannula 108. The inlet area 110 may include a pump housing 103 with a peripheral wall 111 extending about a rotation axis of the impeller blades, positioned radially outward of the inner surface with respect to the rotation axis of the impeller. The impeller may be rotatably coupled to the pump 101 at the inlet area 110 adjacent to the blood exhaust apertures 117 formed in the peripheral wall 111 of the pump housing 103. The pump housing 103 may be composed of a metal in accordance with some implementations. The atraumatic extension 102, also referred to as a “pigtail,” may be connected to the distal end 107 of the cannula 108 and may assist with stabilizing and/or positioning the blood pump assembly 100 into the correct position in the heart. The atraumatic extension 102 may be configurable from a straight to a partially curved configuration. The atraumatic extension 102 may be composed, at least in part of a flexible material, and may have dual stiffness. It should be appreciated that some embodiments of the pump assembly may not include atraumatic extension 102.

The cannula 108 may have a shape which matches (or is similar to) the anatomy of the right ventricle of a patient. In the exemplary embodiment shown in FIG. 1A, the cannula has a proximal end 105 arranged to be located near the patient's inferior vena cava, and a distal end 107 arranged to be located near the pulmonary artery. The cannula 108 may include a first segment S1 extending from the inflow area to a point B between the inlet area 110 and the outlet area 106. The cannula 108 may also include a second segment S2 extending from a point C, which is between the inlet area 110 and the outlet area 106, to the outlet area 106. In some implementations, points B and C may be located at the same location along cannula 108. The first segment S1 of the cannula may form an ‘S’ shape in a first plane. In some implementations, segment S1 can have curvatures between 30 degrees and 180 degrees. The second segment S2 of the cannula may form an ‘S’ shape in a second plane. In some implementations, segment S2 can have curvatures between 30 degrees and 180 degrees (e.g., 40°, 50°, 60°, 70°, 80°, 90°, 100°, 110°, 120°, 130°, 140°, 150°, 160°, or 170°). The second plane can be different from the first plane. In some implementations, the second plane may be parallel or identical to the first plane.

Although shown with an ‘S’ shape, it will be appreciated that other implementations of the blood pump assembly may be formed with other shapes (e.g., a ‘U’ shape), or with no shape at all when outside the body. In such implementations, the cannula may be formed of a flexible material such that the cannula may bend during insertion and achieve the desired shape once inside the heart of the patient.

In some implementations, the blood pump assembly 100 may be inserted percutaneously through the internal jugular vein, though the right atrium and into the right ventricle. When properly positioned, the blood pump assembly 100 may deliver blood from the inlet area 110, which sits inside the patient's right atrium, through the cannula 108, to the blood exhaust apertures 117 of the pump housing 103 positioned in the pulmonary artery. Alternatively, in some implementations the blood pump assembly 100 may be inserted percutaneously through the femoral artery and into the left ventricle to deliver blood from the left ventricle into the aorta.

FIG. 1B shows that blood pump assembly 100 may form part of a cardiac support system 120. Cardiac support system 120 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of blood pump assembly 100. During operation, purge subsystem 150 may be configured to deliver a purge fluid to blood pump assembly 100 through catheter 112 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between blood pump assembly 100 and controller 130. Plug 170 may connect catheter 112, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition blood pump assembly 100 in the patient's heart (e.g., by holding a position of the pump assembly relative to the patient).

As shown in FIG. 1B, in some embodiments, the cardiac support system 120 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 120 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to blood pump assembly 100. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 may include a flexible rubber diaphragm that provides the additional filling volume by means of an expansion chamber. Infusion filter 158 may help prevent bacterial contamination and air from entering catheter 112. Sidearm 159 may provide a fluidic connection between infusion filter 158 and plug 170. Although shown as having separate purge tubing and connector cable, it will be appreciated that in some embodiments, the cardiac support system 120 may include a single connector with both fluidic and electric lines connectable to the controller 130.

During operation, controller 130 may be configured to control the electrical power delivered to the motor of the blood pump assembly 100 (e.g., via connector cable 160), thereby controlling the speed of the motor. Blood pump assembly 100 may include a current sensor (not shown) configured sense motor current associated with an operating state of the motor, and controller 130 may be configured to receive the output of the current sensor as a motor current signal. Controller 130 may be configured to detect an obstruction event based, at least in part, on the motor current signal and a pump speed signal based on a measured speed of the pump, as described in more detail herein. The current sensor may be included in controller 130 or may be located along any portion of the connector cable 160 between controller 130 and the motor. Additionally or alternatively, the current sensor may be included in the motor and controller 130 may be configured to receive the motor current signal via a data line coupled to controller 130 and the motor.

In some embodiments, controller 130 may be configured to receive measurements from one or more pressure sensors (not shown) included as a portion of blood pump assembly 100 and purge disc 154. Controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter 112 and plug 170. Sensor cables (not shown) within catheter 112, connector cable 160, and plug 170 may provide an electrical connection between components of the blood pump assembly 100 (e.g., one or more pressure sensors) and controller 130. Motor cables (not shown) within catheter 112, connector cable 160, and plug 170 may provide an electrical connection between the motor of the blood pump assembly 100 and controller 130. During operation, controller 130 may be configured to receive measurements from one or more pressure sensors of the blood pump assembly 100 through the sensor cables (e.g., optical fibers) and to control the electrical power delivered to the motor of the blood pump assembly 100 through the motor cables. By controlling the power delivered to the motor of the blood pump assembly 100, controller 130 may be operable to control the speed of the motor.

Various modifications can be made to cardiac support system 120 and one or more of its components. For instance, one or more additional sensors may be added to blood pump assembly 100. In another example, a signal generator may be added to blood pump assembly 100 to generate a signal indicative of the rotational speed of the motor of the blood pump assembly 100. As another example, one or more components of cardiac support system 120 may be separated. For instance, display 140 may be incorporated into another device in communication with controller 130 (e.g., wirelessly or through one or more electrical cables).

As described herein, a cardiac support system may include one or more sensors configured to sense quantities associated with operation of a heart pump. For instance, a current sensor may be configured to sense a motor current signal associated with operation of the motor of the heart pump, a speed sensor may be configured to sense a pump speed associated with operation of the rotor of the heart pump, and one or more pressure sensors may be configured to detect a pressure near an inlet of the heart pump where blood is ingested and/or an outlet of the heart pump where blood is expelled. For instance, when a right heart cardiac support device is positioned properly, the outlet of the heart pump may be positioned within the pulmonary artery of a patient's heart, and a pressure sensor arranged near the outlet of the heart pump may measure the pressure within the pulmonary artery. The pressure signal may be used, at least in part, to determine a blood flow rate through the heart pump when in operation. For instance, the pressure signal may be used in combination with a motor current signal received from a motor current sensor (not shown) and a set of stored values to determine a flow rate of blood through the heart pump. For a right heart cardiac support device, the differential pressure between the right atrium and the pulmonary artery may also indirectly be determined based on the pressure signal measuring the pressure in the pulmonary artery and the set of stored values. Alternatively, a differential pressure between the right atrium and the pulmonary artery may be determined using multiple pressure sensors, one located at an inflow region of the heart pump and another located at an outflow region of the heart pump. In some embodiments, one or more pressure sensors associated with a heart pump may be implemented as an optical sensor configured to measure a pressure within the patient's heart using optical signals.

Suction events in which the inlet of a heart pump is obstructed during operation of the pump may be characterized by a decrease in blood flow through the pump and/or a decrease in the motor current signal. Indications of these signals may be displayed on a display (e.g., display 140) of a controller (e.g., controller 130) of the cardiac support system, and a user may monitor the signals on the display to determine when the patient is not receiving adequate support from the heart pump. The inventors have recognized and appreciated that providing an alarm or other indication of a suction event on the display may alert the user that corrective action should be taken to address the suction event. Some existing techniques for detecting suction events during operation of a heart pump are based only on detecting changes in the pulsatility of the motor current signal. Although the pulsatility of the motor current signal may be a reasonably reliable metric to detect suction events in some instances (e.g., when the heart pump is placed in the left side of the heart), in some other instances (e.g., when the heart pump is placed in the right side of the heart) the cardiovascular characteristics do not exhibit the same degree of pulsatility changes.

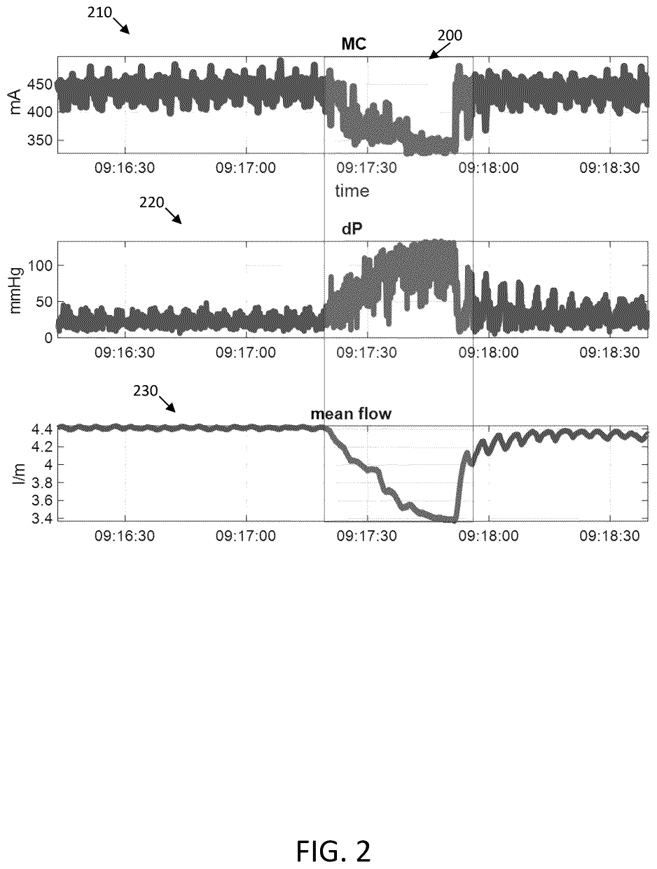

FIG. 2 illustrates example plots for a motor current signal 210 and a pressure signal 220 (e.g., a differential pressure (dP) signal) that may be sensed during operation of a heart pump, in accordance with some embodiments. FIG. 2 also shows a pump flow signal 230 determined based, at least in part, on the pressure signal 220. For instance, pump flow signal 230 may be determined based on the pressure signal 220 and stored values that associate pressure values with pump flow values. In the example shown in FIG. 2, the pump flow signal 230 represents the mean blood flow through the heart pump. Time window 200 highlights a time period during which a suction event may be detected, in accordance with some embodiments.

As shown in FIG. 2, within time window 200, the motor current signal 210 shows a decrease relative to a baseline motor current signal value established prior to time window 200. Within time window 200, the pressure signal 220 shows an increase relative to a baseline pressure signal value established prior to time window 200. The increase in pressure signal 220 within time window 200 is reflected as a decrease in the pump flow signal 230 relative to a baseline pump flow value prior to time window 200.

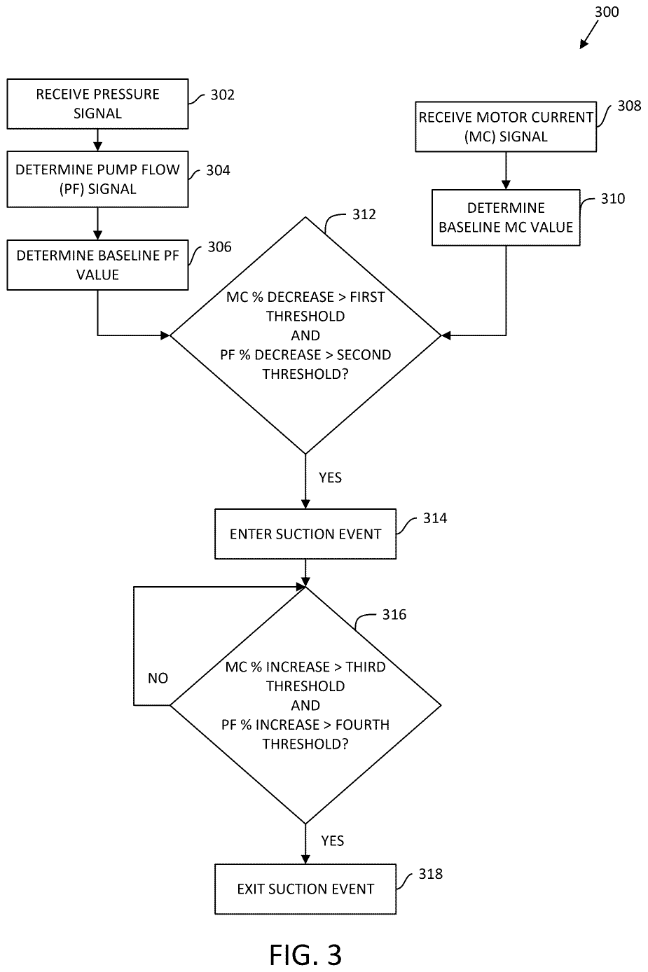

In some embodiments, a time-based (e.g., trending) analysis of multiple signals may be used to detect a suction event. FIG. 3 illustrates a process 300 for detecting a suction event associated with operation of a heart pump, in accordance with some embodiments. In process 300, a pressure signal 302 (e.g., a differential pressure signal) may be received from a sensor (e.g., an optical pressure sensor). Process 300 may then proceed to act 304, where a pump flow signal may be determined based, at least in part, on the received pressure signal. For example, in some embodiments, the pump flow signal may represent a mean flow through the pump (e.g., in liters/minute) and may be determined based on the received pressure signal and stored values associating pressure signal values and pump flow values. In some embodiments, the pump flow signal may be determined based, at least in part, on a current pump speed (e.g., P-level) of the heart pump. Process 300 may then proceed to act 306, where a baseline pump flow value may be determined based on the pump flow signal. In some embodiments, determining the baseline pump flow value is determined after waiting a particular amount of time (e.g., 5 seconds, 10 seconds, 20 seconds, 30 seconds, 40 seconds, 1 minute) after the beginning of pump operation and/or after a change in pump speed (e.g., by changing the P-level of the pump). In some embodiments, the baseline pump flow value may be calculated as a mean flow value determined within one or more time windows (e.g., one or more two second time windows) of the pump flow signal. As described in more detail below, changes in the pump flow value of the pump flow signal relative to the baseline pump flow value may be used, at least in part, to detect a suction event according to some embodiments.

In act 308, a motor current signal (e.g., as sensed by a current sensor associated with a motor of the heart pump) may be received. Process 300 may then proceed to act 310, where a baseline motor current value is determined. Similar to the baseline pump flow value, determining the baseline motor current value may be determined after waiting a particular amount of time (e.g., 5 seconds, 10 seconds, 20 seconds, 30 seconds, 40 seconds, 1 minute) after the beginning of pump operation and/or after a change in pump speed (e.g., by changing the P-level of the pump). In some embodiments, the baseline motor current value may be calculated as a mean motor current value determined within one or more time windows (e.g., one or more two second time windows) of the pump flow signal. As described in more detail below, changes in the value of the motor current signal relative to the baseline motor current value may be used, at least in part, to detect a suction event according to some embodiments.

After establishing a baseline pump flow value in act 306 and a baseline motor current value in act 310, process 300 may proceed to act 312, where it is determined whether (1) a current value of the motor current signal has decreased a first threshold amount relative to the baseline motor current value and (2) a current value of the pump flow signal has decreased a second threshold amount relative to the baseline pump flow value. In some embodiments, the current value of the motor current signal and/or the current value of the pump flow signal may be determined within a time window (e.g., a two second time window). In some embodiments, the first threshold and the second threshold may be specified as a percentage decrease from the corresponding baseline value. For instance, the first threshold or the second threshold may be a 10% decrease, a 15% decrease, a 20% decrease or any other suitable value. The first threshold and the second threshold may be determined in any suitable way. For example, the first threshold and/or the second threshold may be empirically determined to minimize a number of false positives. It should be appreciated that the first and second thresholds may be the same or different. Additionally, the first threshold and/or the second threshold may be different for different pump speeds (e.g., P-levels). Accordingly, each time the pump speed is changed, a new first threshold and/or second threshold may be determined (e.g., using stored threshold values for the corresponding pump speed at which the pump is operating). If it is determined in act 312 that there has been a sufficient decrease in both the motor current value and the pump flow value relative to their corresponding baseline values, process 300 may proceed to act 314, where a suction event is detected and the heart pump may be considered to be in a “suction state.” Otherwise, if it is determined in act 312 that at least one of the conditions is not met, the motor current signal and the pump flow signal may continue to be monitored until it is determined in act 312 that both conditions for detecting a suction event are met.

In response to detecting a suction event, an action may be performed to alert a user of the cardiac support system that a suction event has been detected. For example, a controller of the cardiac support system may be configured to output one or more alarms on a display associated with the controller. In some embodiments, outputting one or more alarms may include outputting a visual indication of the suction event on the display, an audio indication of the suction event, a combination of a visual indication and an audio indication of the suction event, or any other suitable alarm(s) to alert the user that a suction event has been detected.

As described herein, a suction event may be reversable such that, if a healthcare professional or other user performs a corrective action (e.g., moving the heart pump to a new position) the suction event may be resolved and the patient may again be provided with adequate cardiac support. Accordingly, some embodiments include exit criteria that enable a cardiac support device in a suction event state to exit the suction event state and resume normal operating behavior. An example of exit criteria in accordance with some embodiments is shown as act 316 in process 300. After a suction event has been detected in act 314, process 300 may proceed to act 316, where it is determined whether (1) a current motor current value has increased from the motor current value at which the suction event was detected more than a third threshold amount (e.g., a 10% increase from the motor current value during suction) and (2) a current pump flow value has increased from the pump flow value at which the suction event was detected more than a fourth threshold amount (e.g., a 12% increase from the pump flow value during suction). In some embodiments, the third threshold and the fourth threshold may be specified as a percentage increase from the corresponding value when the suction event was detected. For instance, the third threshold or the fourth threshold may be a 10% increase, a 15% increase, a 20% increase or any other suitable value. The third threshold and the fourth threshold may be determined in any suitable way. For example, the third threshold and/or the fourth threshold may be empirically determined to minimize a number of false positives. It should be appreciated that the third threshold and the fourth threshold may be the same or different. Additionally, the third threshold and/or the fourth threshold may be different for different pump speeds (e.g., P-levels). Accordingly, each time the pump speed is changed, a new third threshold and/or fourth threshold may be determined (e.g., using stored threshold values for the corresponding pump speed at which the pump is operating).

If it is determined that both exit conditions are met in act 316, process 300 may proceed to act 318, where the suction state of the cardiac support system may be exited. If it is determined in act 316 that at least one of the exit conditions is not met, the motor current signal and the pump flow signal may continue to be monitored until it is determined in act 316 that both conditions for detecting a suction event are met. In response to exiting a suction event, an action may be performed to inform the user of the cardiac support system that the suction event has been cleared. For example, a controller of the cardiac support system may be configured to disable one or more alarms presented on a display associated with the controller when the suction event was detected (e.g., by removing a visual indication of the suction event alarm(s) from the user interface).

As described herein, some embodiments of the present disclosure use a trending analysis (e.g., percentage decrease or percentage increase of a signal value over time) to detect the start of and/or exit from a suction event. However, establishing one or more suitable baseline values to use in such a trending analysis may take an amount of time (e.g., 20 seconds, 30 seconds, 40 seconds, 1 minute) following initial operation of the cardiac support system and/or when changing the pump speed, during which suction events may not be detected. In some embodiments, during the time in which one or more suitable baseline values have not yet been established, an alternate measure (e.g., absolute value of a differential pressure signal) may be used to detect suction events. Although the alternate measure may have reduced accuracy compared with the suction event entry/exit criteria described in connection with process 300 of FIG. 3, using an alternate measure may provide some ability to detect suction events during the relatively short time period during which the trending analysis may not be applied.

Different types of suction events may be addressed using different corrective actions. For example, biomaterial (e.g., tissue) in the inlet/outlet may result in a quick suction event that may be resolved by the user adjusting the position of the heart pump. Another type of suction event may be a volume suction event in which the volume in the central venous system is slowly depleted. A different corrective action may be better at clearing a volume suction event compared with a quick suction event. The inventors have recognized and appreciated that different types of suction events may have different characteristics or “signatures” that can be identified based on an analysis of one or more of the signals described herein. In some embodiments, rather than simply detecting whether a suction event has occurred, information from one or more signals associated with a heart pump may be used to determine a type of suction event (e.g., quick suction event, volume suction event). For example different windows and/or multiple windows may be used to identify a type of suction event. In some embodiments, a recommended correction action (e.g., provide the patient with more liquid, slow the pump speed, reposition the pump, etc.) may be provided to a user (e.g., on a user interface) based, at least in part, on the type of suction event that is detected.

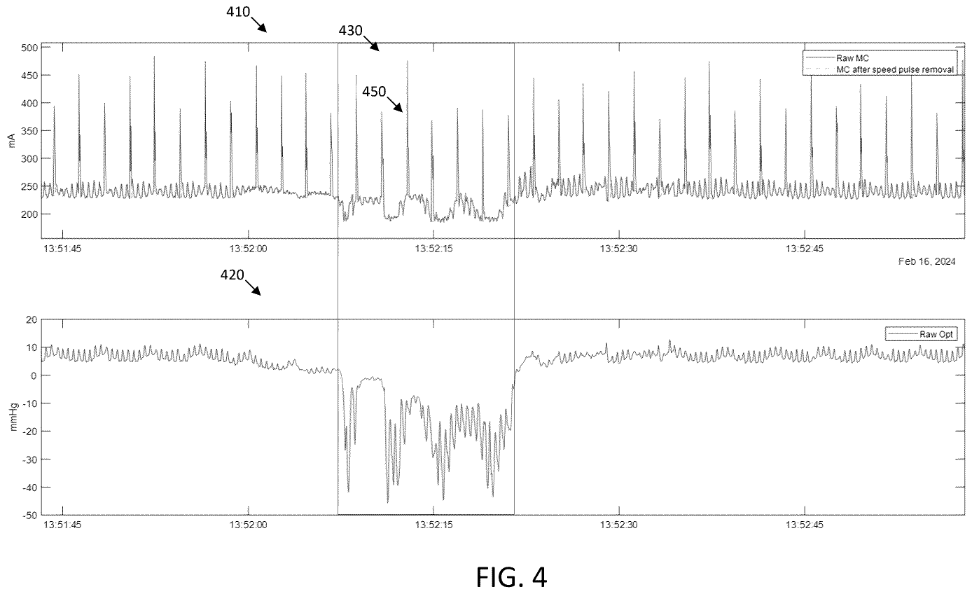

FIG. 4 illustrates example plots for a motor current signal 410 and a pressure signal 420 (e.g., an optical pressure signal) that may be recorded during operation of a heart pump, in accordance with some embodiments. Time window 430 highlights a time period during which a suction event may be detected, in accordance with some embodiments. As shown in FIG. 4, within time window 400, the motor current signal 410 shows a decrease relative to a baseline motor current signal value established prior to time window 430. Within time window 430, the pressure signal 420 shows a decrease relative to a baseline pressure signal value established prior to time window 430. As shown in FIG. 4, at certain pump speeds the motor current signal 410 may exhibit sharp pulses 450, which may make it challenging to detect suction events. As discussed in further detail below, in some embodiments, one or more of the sensed signals may be filtered to reduce or remove such sharp pulses prior to detecting suction events.

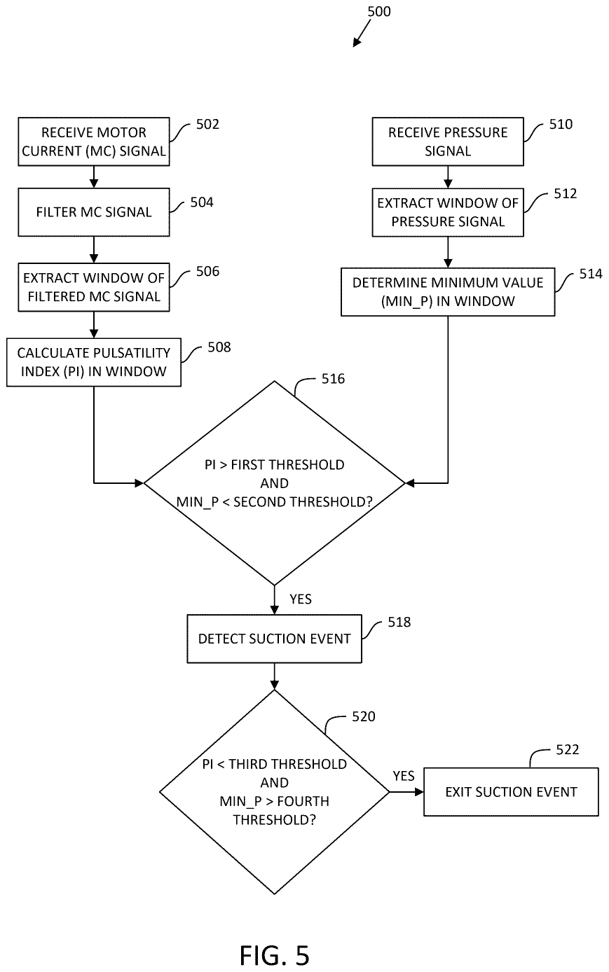

In some embodiments, a suction event may be detected without using a trending analysis as described in connection with process 300 of FIG. 3. FIG. 5 illustrates a process 500 for detecting a suction event associated with operation of a heart pump, in accordance with some embodiments. Process 500 may begin in act 502, where a motor current signal associated with operation of a heart pump may be received. Process 500 may then proceed to act 504, where one or more filters may be applied to the motor current signal to generate a filtered motor current signal. For instance, as described herein, the motor current signal may be filtered to remove or reduce sharp pulses that may be present at certain pump speeds. In some embodiments, applying a filter may include applying an infinite impulse response (IIR) filter. Process 500 may then proceed to act 506, where a time window of the filtered motor current signal may be extracted for analysis. The extracted time window may have any suitable length. For example, the time window may be 2 seconds, 4 seconds, 6 seconds, 8 seconds, etc. As will be appreciated, different time windows may capture different phenomena associated with suction events, which may occur more frequently when the blood volume near the inlet of the heart pump is at a minimum. For example, in embodiments where the heart pump is positioned in the left heart, the extracted time window may be selected, based at least in part, on the heart rate of the patient and/or a timing of when the left ventricle is ending contraction and the volume of blood in the left ventricle (near the inlet of the heart pump) is at a minimum during the cardiac cycle. In such embodiments, it may be preferable to examine a shorter time window that coincides with the cardiac cycle. In embodiments where the heart pump is positioned in the right heart, the extracted time window may be selected based on the respiratory cycle of the patient. For example, the volume of blood in the vena cava and/or the right atrium (near the inlet of the heart pump) may be lowest at a point in the respiratory cycle associated with central venous return. In such embodiments, it may be preferable to examine a longer time window that coincides with the respiratory cycle. Process 500 may then proceed to act 508 where a pulsatility index (PI) is calculated within the extracted time window. For example, in some embodiments the PI may be calculated according to the following formula:

$$PI = ( max ( MC\_win ) - min ( MC \_win ) ) / mean ( MC \_win ) ,$$

where MC_win is the extracted time window of the filtered motor current signal.

As described in more detail below, the pulsatility index determined in act 508 may be used, at least in part, to detect a suction event according to some embodiments. In act 510 of process 500, a pressure signal (e.g., an optical pressure signal) may be received. An example of an optical pressure signal is shown in FIG. 4. Process 500 may then proceed to act 512, where a time window of the pressure signal may be extracted for analysis. The extracted time window may have any suitable length. For example, the time window may be 2 seconds, 4 seconds, 6 seconds, 8 seconds, etc. Process 500 may then proceed to act 514, where a minimum value of the pressure signal (Min_p) within the extracted time window is determined. As described in more detail below, the minimum pressure value Min_p determined in act 514 may be used, at least in part, to detect a suction event according to some embodiments. It should be appreciated that in some embodiments, the pressure signal received in act 510 may be processed (e.g., using one or more filters) prior to determining the minimum pressure value Min_p.

After determining the pulsatility index in act 508 and the minimum pressure value in act 514, process 500 may proceed to act 516, where it is determined whether (1) the pulsatility index is greater than a first threshold and (2) the minimum pressure value (Min_p) is less than a second threshold. The first threshold and the second threshold may be determined in any suitable way. For example, the first threshold and/or the second threshold may be empirically determined to minimize a number of false positives. It should be appreciated that the first threshold and/or the second threshold may be different for different pump speeds (e.g., P-levels). Accordingly, each time the pump speed is changed, a new first threshold and/or second threshold may be determined (e.g., using stored threshold values for the corresponding pump speed at which the pump is operating). If it is determined in act 516 that both conditions are satisfied, process 500 may proceed to act 514, where a suction event is detected and the heart pump may be considered to be in a “suction state.” Otherwise, if it is determined in act 516 that at least one of the conditions is not met, the pulsatility index and the minimum pressure value may continue to be monitored until it is determined in act 516 that both conditions are met.

In response to detecting a suction event in act 518, an action may be performed to alert a user of the cardiac support system that a suction event has been detected. For example, a controller of the cardiac support system may be configured to output one or more alarms on a display associated with the controller. In some embodiments, outputting one or more alarms may include outputting a visual indication of the suction event on the display, an audio indication of the suction event a combination of a visual indication and an audio indication of the suction event, or any other suitable alarm(s) to alert the user to the detected suction event.

As described herein, a suction event may be reversible if a user takes appropriate corrective action to resolve the suction event. Accordingly, process 500 may proceed to act 520, where it is determined whether (1) the pulsatility index (PI) is less than a third threshold and (2) the minimum pressure value (Min_p) is greater than a fourth threshold. When it is determined that both conditions have been satisfied, process 500 may proceed to act 522, where it may be determined that the suction exit criteria have been satisfied and an indication of the suction event exit may be provided to a user via the user interface associated with the cardiac support system. For instance, a suction event alarm displayed on the user interface during the suction event may be removed from the user interface.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of detecting a suction event associated with operation of a heart pump, the method comprising:
receiving a motor current signal associated with a motor of the heart pump;
receiving a pressure signal associated with the heart pump;
determining a pump flow signal based on the pressure signal;
detecting, using a computer processor, a suction event based, at least in part on:
a first value of the motor current signal within a first time window being a first threshold amount less than a baseline motor current signal value; and
a first value of the pump flow signal within the first time window being a second threshold amount less than a baseline pump flow value; and
displaying, when a suction event is detected, an indication of the suction event on a user interface associated with the heart pump.

2. The method of claim 1, wherein the pressure signal comprises a differential pressure signal across at least one valve of a heart of a patient.

3. The method of claim 2, wherein determining the pump flow signal based on the pressure signal comprises:
accessing stored data associating pump flow values to differential pressure signal values; and
determining the pump flow signal based on stored data.

4. The method of claim 1, wherein the first value of the motor current signal within the first time window is a mean value of the motor current signal within the first time window.

5. The method of claim 1, wherein the first value of the pump flow signal within the first time window is a mean flow value within the first time window.

6. The method of claim 1, wherein the heart pump is positioned across a valve in a right side of a heart of a patient.

7. The method of claim 1, further comprising:
determining, based on the motor current signal, the baseline motor current signal value, wherein the baseline motor current signal value is determined within at least one second time window before the first time window; and
determining, based on the pump flow signal, the baseline pump flow value, wherein the baseline pump flow value is determined within the at least one second time window.

8. The method of claim 1, wherein the first threshold amount is a first threshold percentage of the baseline motor current signal value.

9. The method of claim 1, wherein the second threshold amount is a second threshold percentage of the baseline pump flow value.

10. The method of claim 1, further comprising:
setting the first threshold amount and the second threshold amount based, at least in part, on a speed of the heart pump.

11. The method of claim 1, further comprising:
detecting, using the computer processor, whether one or more exit criteria for the suction event are satisfied; and
displaying, when the one or more exit criteria for the suction event are satisfied, an indication that the suction event has ended on the user interface associated with the heart pump.

12. The method of claim 11, wherein detecting whether one or more exit criteria for the suction event are satisfied comprises detecting that the one or more exit criteria are satisfied when:
a second value of the motor current signal within a second time window is more than a third threshold amount greater than the first value of motor current signal; and
a second value of the pump flow signal within the second time window is more than a fourth threshold amount greater than the first value of the pump flow signal.

13. The method of claim 12, wherein the third threshold amount is a percentage of the first value of the motor current signal.

14. The method of claim 12, wherein the fourth threshold amount is a percentage of the first value of the pump flow signal.

15. The method of claim 12, wherein the first threshold amount and the third threshold amount are different.

16. The method of claim 12, wherein the second threshold amount and the fourth threshold amount are different.

17. The method of claim 11, wherein
displaying, when a suction event is detected, an indication of the suction event on a user interface associated with the heart pump comprises displaying an alarm on the user interface, and
displaying, when the one or more exit criteria for the suction event are satisfied, an indication that the suction event has ended on the user interface associated with the heart pump comprises removing the alarm from the user interface.

18. The method of claim 17, wherein the alarm comprises a visual alarm and/or an audio alarm.

19. A controller for a heart pump system, the controller comprising:
at least one hardware processor configured to:
receive a motor current signal associated with a motor of a heart pump of the heart pump system;
receive a pressure signal associated with the heart pump;
determine a pump flow signal based on the pressure signal;
detect a suction event based, at least in part on:
a first value of the motor current signal within a first time window being a first threshold amount less than a baseline motor current signal value; and
a first value of the pump flow signal within the first time window being a second threshold amount less than a baseline pump flow value; and
display, when a suction event is detected, an indication of the suction event on a user interface associated with the heart pump system.

20-36. (canceled)

37. A heart pump system, comprising:
a heart pump including a motor and a pressure sensor configured to sense a pressure within a portion of a heart of a patient; and
a controller configured to:
determine a pump flow signal based on a pressure signal sensed by the pressure sensor;
detect a suction event based, at least in part on:
a first value of a motor current signal associated with the motor within a first time window being a first threshold amount less than a baseline motor current signal value; and
a first value of the pump flow signal within the first time window being a second threshold amount less than a baseline pump flow value; and
display, when a suction event is detected, an indication of the suction event on a user interface associated with the heart pump system.

38-60. (canceled)
